SEQUENCE LISTING (14 sequences)

SEQ ID NO: 1 - DNA, 31 bp, Artificial Sequence
  Primer 515F
tatggtaattgtgtgccagcmgccgcggtaa

SEQ ID NO: 2 - DNA, 32 bp, Artificial Sequence
  Primer 806R
agtcagtcagccggactachvgggtwtctaat

SEQ ID NO: 3 - DNA, 20 bp, Artificial Sequence
  Primer 27F
agagtttgatcmtggctcag

SEQ ID NO: 4 - DNA, 19 bp, Artificial Sequence
  Primer 1492R
ggttaccttgttacgactt

SEQ ID NO: 5 - protein, 77 aa, Streptococcus salivarius
  Bacteriocin locus 1 ctg1_271
MTTQTMNNFETLDLEALANVEGGNRCKDLIFGGALTGAGAGFTGGMAAFGVTAFPGAFVGAHVGAVAGGLACIGASF

SEQ ID NO: 6 - protein, 581 aa, Streptococcus salivarius
  Bacteriocin locus 1 ctg1_272
MPNQNQWSVFKRLLTYLKPYKFLTFLALTFLLSTTIIKSIIPLVASYFIDHYLHDMNQAASLILIGYYGLYLLQTLVQYLGNLFFARVSYSIVRDIRRDAFANMEKLGMSYFDKTPAGSIVSRLTNDTETISEMFSGILSSFISAIFIFVTTLYTMMILDIRLTGLIVLFLPLIVLLVNLYRKKSVVIIEKTRALLSDINAKLSESIEGIRIIQAFNQEKRLKKEFDAINEEHLIYASRSMSLDSLFLRPAMSLLKLLGYAVLMAYFGYRGIYLGITAGTMYAFIQYINRLFDPLIEVTQNFSTLQTSMVSAGRVFALIDERTYEPEQRDTDAKVTEGNIRFENVSFSYDGKHQILDNISFSVNKGETIAFVGSTGSGKSSIINVFMRFYEFQSGRVLLDGVDIRDYSQAELRKNIGLVLQEPFLYHGTIKSNIAMYQDLTDEEIKAAAEFVDANHFIDKLPEGYDAPVSERGSSFSTGQRQLLAFARTVASQPKILILDEATANIDSETEAIVQNSLAKMRQGRTTIAIAHRLSTIQDANCIYVLDKGRIIEHGSHEELLALGGTYHKMYSLQAGAMEGA

SEQ ID NO: 7 - protein, 585 aa, Streptococcus salivarius
  Bacteriocin locus 1 ctg1_273
LFFKESIIAKLWWFFKLEKRRYIVGILALSLVSVLNLIPPMVMGRVIDAITSGKLTNQILLLNLVYLLLAAIGMYYLRYVWRMYILATSYRLGQIMRSRLFEHFTKMSPSFYQKYRTGDLMAHATNDINSLTRLAGGGVMSAVDASITALVTLITMFFSISWQMTLVAVLPLPFMAFATSRLGRKTHKAFGESQAAFSELNNKVQESVSGIKLTKSFGYQEAELQSFQETNKMTFKKNMHTMKYDSLFDPLVLLFVGSSYVLTLLVGAFMIQAGQITVGNLVTFITYLDMLVWPLMAIGFLFNITQRGNVSYQRIENLLEQESDVQDPKHPLPSIENGRLEYAIDRFAFEDEDTLRDIHFTLDKGQTLGLVGQTGSGKTALVKLLLREHDVNQGAIYLNGHDIRDYRLSDLRSLMGYVPQDQFLFASSILDNVRFGNPDLAFEKVEEATKLAQVYDDIKAMPEGFETIIGEKGVSLSGGQKQRLAMSRAMILDPDILILDDSLSAVDAKTEHAIIDNLKHTRKDKTTIITAHRLSAVVHADLVLVMQNGRIIERGRHEDLLAQGGWYAKTYESQQLEMEGGEVDA

SEQ ID NO: 8 - protein, 358 aa, Streptococcus salivarius
  Lassopeptide ctg2_139
VEKSLTFNQLVKQTYFKILRQFLINLFLFYLLPMLLTAFTNITEKNAGAFTIYFSTASWIYISLILLFSITKNMRNLLEKIKQETIIVYNQSLLGDTNENNSAKLTLIELIETRQKIQEMQATIKKIIQSEKQQKQELMFQVSAAAHDLKTPLTVIQGNAQFLQSMDIPGNIEQCLEDIELASQQLNLYFNQLINYSKTFYNDTSNWESLSSDYLFELFEQEIKLITDKKADIQFSNNLPTSIYLTLNLNLFLRATLNIINNAIDHSQSSYPTIKVNYKFIDNQFYISIWNSDSSFTENLLEKYGLLFYQDKQATNSEQRSNFGIGLAFVKRVAKIHGGQVNLSNCNNGALVTICIPV

SEQ ID NO: 9 - protein, 223 aa, Streptococcus salivarius
  Lassopeptide ctg2_140
MKYKILVIDDDKSILRLVRNVLESKNFVVDTLTSIPDIDICHFIGYDLIILDIMMPHSGIEICRYIREFISVPIIFLTAKNLEEDVVCGIQSGADDYITKPFSIPELVARVQMHIRREVRNNNSNEILQFGPITINKIKEELRVADVHVPTTKREFQLILLLSSNPNKIFSNEELFDYLYPHDSETQGRSITEYVYQVRQKLKPYELNPIKTIWGGGYQWRSL

SEQ ID NO: 10 - protein, 587 aa, Streptococcus salivarius
  Lassopeptide ctg2_142
MKVNVILSKNQIITTDTLIDELVVSEELKTYTADNLLMIVKPHKNSEDIGRFLAKENLNIEMIESNLLELKKNASFVAITKENHFVIGGDFNNHNPIYYLEKDEELFISFYPELIASFTKQKLNRTYFALRIVNFDTVYPFTELTPWDNVKLVNSRKILVSLEGQVKQVFPWRSNGENKTIAGVAGELSKNLNSVLRKYLENQPKISADLSGGLDSTSLCYFCKNLGIELNTVFLSPEDGADSDVVWSNRASRDVSAEHVILPYSSTVTLSTLDPIAIYNALPFGPSESFRYINLARKIIEIGLENDISTHINGHGGDELFGPLSTMAWSLYHSNYPNRIRNLYGFARLNKFNLFKFFKSLTDNTSLSDNLRNLVCQEVEQVDFDIKYSSKWISTLSVESFVTQTARDLITRCATSYAEKDIESYSDDKSIHKILEEIESHTLLQRDLNAMVPTDKYFRFLSPYTNYDIVESALKYNIDERFNAKVTKPMLAEIRPSSMSLEYFQRRDKGEYSKPTFTEYSKRKKILQKIFSEECLIYKLGLVDRVDLLESLNKFSADGELLENVMRLQIIEFWLRGYCESEGEYEI

SEQ ID NO: 11 - protein, 139 aa, Streptococcus salivarius
  Lassopeptide ctg2_144
MSSPVRMEQRTKLSVKNKITALFCANVSFFLIKLPPKKLSEVIEKLSKNTRKALPKEVESWRTSINSINVRCAGNGCLQRSVAVMLRGIIARRTPDWVSGFQVSPFIAHAWVEVDGIPIGEEMDLSNFQKILFVKGGQR

SEQ ID NO: 12 - protein, 235 aa, Streptococcus salivarius
  Lassopeptide ctg2_145
MIKIENVSKAFREKIVLSKISFTANEGEVTALIGPNGSGKSTLLKILLGLISTDEGRATFDGKNYGELGKYPFRYVGTFLDSFQPNPTRTAYNHLRWIALTSGISKQRCLECLKIVGLQDVGNKKIKDYSLGMKQRLGLATAILANPKILILDEPINGLDPDGIRWVREFLREFVKDDKIVLITSHYMNELELTVDKIVGLSNGKVVIDGSSKDVLEEYGSFEEAYFRSVRNEMR

SEQ ID NO: 13 - protein, 580 aa, Streptococcus salivarius
  Lassopeptide ctg2_148
MDSQQHSEVSFREFLRLLKPHKISISIALIIALIASTLSILQPILLSKIIDNINNTILKKTVILFSMIVLSSAILNSIKQYILESISENLVNSLRIQIVNRSIKYKIEVFDTRKIGDLASILSTDTAQLRGILSQGIVELVSQTFTMLFALIMMFYLDIQLFSLSLLAVLSLLICGLLLGKRTRPIAKDLQEVVGELSSELERTLRGVRTIRAFLSENVFVERMSSVVTSATKIGKKVAIFKSIISGFSNIALQIMLIIIIGVGAIRVATGVISIGSLSAFIMYVMLVLTPAAMLGGVLASINEGLGAYSRIKVALELPIEEDINSLDVLEKKENQILQIKKLSFKYNDNFDKNILNKIDISVNGPGVTALVGPSGSGKTTIFELIERFYDFDDGTIELYGNNIYDLPIKVLRSNITFVEQNASIFSGTVLENLQIANKNVSRLDCLHALNLVNLFQDIPSEEILDRVIGESGITLSGGEKQRLSLARALISPNDIILLDETTSNLDSINESKIHQLIKDLSRRKTVLIIAHRLSTIQNADSIYLISEGEVDDCGTHQELLSRNKLYKELVETQFINKET

SEQ ID NO: 14 - protein, 698 aa, Streptococcus salivarius
  Bacteriocin locus 2 ctg4_81
MSKYPYISQVDQRDCGVATLAMILKHYGSSYSLAYLRELAQTSREGTSALGLVEAAKQLGLETQAIRADLDLFKQENLSYPFIVHVVKEGGLQHYYTVFGQIKGQLVIGDPDPSKKVIKMPLEEFSKEWTGVALFFVPGEAYTKYKEDVPGLLSFLPILFRRKGLIAVIVLLSFLVTLVNIIGSYYLQAIIDRLIPQGAYNLLTMISLGLCISYLAQQVFTFFKDYLLHRLGNYLSIAVILPYIKHVLSLPISFFSSRRTGEITSRFGDANTIIDALASTILSIFLDVTIVMTLAVALIVQNQSLFLMTLTVVPIYVLIIVAFYKLFEKENYQLMEANSQVNTAVIDDLRGIETLKSLRVEERRYREIEVKFHDYLKKSLSKAKWQLTQDGLKTGVQLVSNVFILWYGAQLVMEGQLSAGQLITYNMLLNYFTTPLINIINLQSKIQQAKVANNRLQEVYVVDKEEKGKLRELSFKQLAFKGVSHRFSYQQETLSKIDLTIHKGEKIALMGKSGSGKTTLAKMLSGYYTKSSGRVTLDKDAISHAELRQLVTYVPQQTYVFTGTILENLLLGFEGEVDEKKLLKVCQQADILEDIQKMPLGFQTQVSEDGGLSGGQKQRLAIARALLSKQPILIFDEATSGLDSDTESRVMANLAKIKRTMIFIAHRNSVRQHVSRVVTMVSGQIESDSPNFNLFQFI

(19) United States
(12) Patent Application Publication
(10) Pub. No.: US 2023/0407241 A1
(43) Pub. Date: Dec. 21, 2023

(54) A NOVEL PROBIOTIC STREPTOCOCCUS SALIVARIUS STRAIN AND ITS USES

(71) Applicant: Universiteit Antwerpen, Antwerpen (BE)

(72) Inventors: Sarah Lebeer, Antwerp (BE); Ilke De Boeck, Antwerp (BE); Jennifer Jörissen, Antwerp (BE); Irina Spacova, Antwerp (BE)

(73) Assignee: Universiteit Antwerpen, Antwerpen (BE)

(21) Appl. No.: 18/023,805

(22) PCT Filed: Sep. 3, 2021
(86) PCT No.: PCT/EP2021/074362

§ 371 Date: Feb. 28, 2023

(30) Foreign Application Priority Data
Sep. 4, 2020 (EP) 20194562.3

(51) Int. Cl. C12N 1/20; C07K 14/315; A61P 27/16; A61P 37/04; A61P 31/04

(52) U.S. Cl. CPC C12N 1/205; C07K 14/315; C12R 2001/46; A61P 37/04; A61P 31/04; A61P 27/16

(57) ABSTRACT

The present invention is directed to a novel isolated bacterial strain of the Streptococcus salivahus (S. salivahus) species. More in particular, a strain of the S. salivahus species deposited at BCCM under accession number LMG P-31813. Further, the present invention relates to a composition comprising an isolated bacterial strain of the S. salivahus species. In another aspect, the present invention further relates to the isolated bacterial strain according to the present invention or the composition according to the present invention for use in immunomodulation and/or in the treatment and/or prevention of infections of diseases.

### FIELD OF THE INVENTION

The present invention is directed to a novel isolated bacterial strain of the Streptococcus salivarius (S. salivarius) species, said strain deposited at BCCM under accession number LMG P-31813. Further, the present invention relates to secondary metabolites produced by the isolated bacterial strain and to a composition comprising the isolated bacterial strain and/or secondary metabolites according to the present invention. Further, the present invention relates to the use of the isolated bacterial strain, the secondary metabolites or the composition described herein.

### BACKGROUND TO THE INVENTION

Ear, Nose and Throat (ENT) conditions or diseases usually originate from a fungal, bacterial or viral infection in the upper tracts of the respiratory system; examples of such infections are some forms of otitis, sinusitis and/or nasal polyposis: usually the treatment of such forms is performed by using topical or oral antibiotics or anti-inflammatory agents. ENT conditions include debilitating conditions impacting the airways, voice, hearing, speech and/or sinuses.

Among the various ENT conditions and diseases, there is Otitis Media (OM). OM refers to a group of inflammatory diseases of the middle ear caused by viral or bacterial infections. Otitis Media with Effusion (OME) in specific is characterized by the presence of middle ear effusion (MEE) behind an intact tympanic membrane in the absence of other signs or symptoms of acute inflammation (e.g, pain or fever) (1). Chronic OME, lasting 3 months or more, is typically treated by surgical placement of ventilation tubes into the tympanic membrane under anesthesia. However, every operation comes with a risk and there is evidence both supporting and rejecting this surgical procedure (2, 3). Hence, the use of ventilation tubes remains a contentious issue, highlighting the need for alternatives.

Middle ear health is closely associated with upper respiratory tract (URT) health. Bacteria normally resident in the nasopharynx, such as nontypeable Haemophilus influenzae, Streptococcus pneumoniae and Moraxella catarrhalis (the classic otopathogens (4)), have been found to form biofilms in the middle ear of OME patients. These biofilms were not found on the healthy middle ear mucosa of patients undergoing cochlear implantation, an important control group allowing access to non-inflamed and non-infected middle ears. These classic otopathogens are also present in the URT of healthy individuals. They are therefore referred to as pathobionts or commensal bacteria with a pathogenic potential in an immunocompetent host. Recent microbiome data also point towards other pathobionts potentially involved in OME such as Alloiococcus otitis and Corynebacterium otitidis (previously known as Turicella otitidis).

In addition to immune system controls, pathobionts are controlled by beneficial bacteria present in the same habitat, through either direct interaction or immunomodulation. Consequently, a loss or decrease in abundance of these beneficial bacteria can allow pathobionts to overgrow, migrate to neighboring sites, and express their virulence traits. Long-term perturbation of the microbiota has been associated with several chronic inflammatory diseases (5) and is also hypothesized to underlie chronic OME.

Preventing such perturbation or restoring a perturbed microbiota through addition of beneficial bacteria could be a valuable method for OME prophylaxis and could reduce the need for surgical intervention. Such a probiotic approach (6) is widely used for the gastrointestinal tract but underexplored for respiratory health or the prevention and treatment of otitis media (7). A few bacterial species have been described so far for use in the treatment of infection of the respiratory tract. For example, EP2555785 describes the use of the Streptococcus salivarius strain with access number DSM 23307 in the treatment of chronic infections of the respiratory tract, more specifically the upper respiratory tract. DSM 23307 is described to adhere to HEp-2 cells, and to produce bacteriocins able to inhibit the growth of S. pneumoniae and S. pyogenes. These features make the strain which is currently, comprised in marketed Rinogermina® probiotic nasal spray, suitable for treating bacterial and/or fungal infections of the upper respiratory tract.

In light of all the above, it appeared that an understanding of the microorganisms causing OME and those protective against OME could help develop treatment methods alternative to antibiotics and surgery. In addition, there is a strong need to isolate new non-pathogenic strains that show probiotic activity in the treatment of respiratory infections, such as for example otitis media with effusion.

In the present application, a novel Streptococcus salivarius strain was identified with a protective effect against respiratory infections, in particular against otitis media with effusion.

### SUMMARY OF THE INVENTION

The present invention is based on the identification of a novel isolated strain of the Streptococcus salivarius (S. salivarius) species. Said strain has been deposited with the Belgian Co-ordinated Collection of Micro-organisms (BCCM) on May 28, 2020 with accession number LMG P-31813. The strain is herein further also indicated with AMBR158.

In accordance with a first aspect, the present invention relates to an isolated bacterial strain of the Streptococcus salivarius (S. salivarius) species, said strain deposited at BCCM under accession number LMG P-31813.

In accordance with an embodiment of the present invention, the bacteria of the isolated bacterial strain are in suspension, freeze-dried, spray-dried, in live or inanimate/postbiotic form, provided the active components are not disrupted.

In accordance with a further aspect, the present invention relates to secondary metabolites, also referred herein as bacteriocins, produced by the isolated bacterial strain of the Streptococcus salivarius (S. salivarius) species of the present invention.

In accordance with a further aspect, the present invention relates to a composition comprising an isolated bacterial strain of the Streptococcus salivarius (S. salivarius) species, or secondary metabolites of the Streptococcus salivarius (S. salivarius) strain of the present invention.

In accordance with an embodiment of the present invention, the composition comprises one or more pharmaceutically acceptable excipients, aromatizing agents or carriers.

In accordance with an embodiment of the present invention, the composition comprises an amount of bacteria in the range between 10³ to 10¹¹ CFU for each gram of the composition.

In a further aspect, the present invention relates to the isolated bacterial strain, the secondary metabolites or the composition according to the invention for use as a medicament in human or veterinary medicine.

In a further aspect, the isolated bacterial strain, the secondary metabolites or the composition according to the present invention are for use in the treatment and/or prevention of infections and/or inflammatory diseases. In a further embodiment, the infections and/or inflammatory diseases are selected, but not limited to, from upper respiratory tract infections; ear, nose, and throat (ENT) infections; oral cavity infections; caries; sinusitis; nasal polyposis; acute otitis media; recurrent acute otitis media; otitis media with effusion; chronic suppurative otitis media; mastoiditis; halitosis; respiratory infections associated with cystic fibrosis. In an even more preferred embodiment, the isolated bacterial strain, the secondary metabolites or the composition of the present invention is for use in the treatment of otitis media with effusion.

In another aspect, the isolated bacterial strain, the secondary metabolites or the composition according to the present invention are for use in immunomodulation; in particular wherein the isolated bacterial strain, the secondary metabolites or the composition is used as an adjuvant to promote an immune response during vaccination or as an immune modulating agent to prevent allergy or reduce allergy symptoms.

In a further aspect, the present invention relates to the use of the isolated bacterial strain, the secondary metabolites, or the composition according to the present invention in the personal hygiene industry, cleaning industry, air purification, or the production of personal care/consumer/cosmetic products. In an even further embodiment of the invention, the use of the isolated bacterial strain, the secondary metabolites or the composition according to the present invention is provided in the oral hygiene industry; in particular in the production of oral hygiene personal care products.

In another aspect, the present invention provides the use of the isolated bacterial strain, the secondary metabolite or the composition according to the present invention as a probiotic. In a further embodiment, the use of the isolated bacterial strain, the secondary metabolite or the composition according to the present invention is provided in the food industry; in particular in the production of dairy and non-dairy fermentation products, dietary supplements, dietary food additives and/or nutraceuticals.

In accordance with a further embodiment, the present invention relates to the isolated bacterial strain, the secondary metabolites or composition or the use thereof according to the present invention, wherein the bacterial strain, the secondary metabolites or the composition is in any form suitable to be administered topically, orally or through the respiratory tract.

Further, the present invention pertains to the bacterial strain, the secondary metabolite or composition or the use thereof according to the present invention, wherein the bacterial strain, the secondary metabolites or the composition is in a pharmaceutical form selected from, but not limited to, a spray, cream, a lotion, a gel, an ointment, a solution, a suspension, an emulsion, a capsule, a tablet, a powder, a granule, drops, inhaler, tooth paste, mouth wash.

Further, the present invention pertains to the bacterial strain, the secondary metabolite or composition or the use according to the present invention wherein the bacterial strain, the secondary metabolite or the composition is formulated to be administered through the respiratory tract by a nebulizer, with or without propellants.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Amplicon sequence variants (ASVs) significantly differentially abundant in the nasopharynx of chronic OME (D) vs controls (H) by ANCOM analysis with stringent correction for multiple testing. Streptococcus 5 can be classified as Streptococcus salivarius, Streptococcus vestibularis or Streptococcus thermophilus, all of which are members of the Streptococcus salivarius group.

FIG. 2: Capacity of Streptococcus species isolated from the upper respiratory tract of healthy children to inhibit classic middle ear pathogens. Mean inhibition of ((A) Haemophilus influenzae, (B), Moraxella catarrhalis, and (C) Streptococcus pneumoniae) as measured through the Spot Assay method. Each point represents the mean value for a different isolate.

FIG. 3: Inhibition of upper respiratory tract and classic and suspected middle ear pathogens (H. influenzae, S. pneumoniae, M. catarrhalis, Streptococcus pyogenes, Staphylococcus aureus, A. otitis and Corynebacterium otitidis) by 7 in-house isolated S. salivarius strains (indicated with AMBR numbers). Isolates were compared to S. salivarius 24SMB and Streptococcus oralis 89a isolated from the Rinogermina® probiotic nasal spray and Hextril mouthwash (0.1% Hexetidine) served as a positive control. For each pathobiont, the tested isolates are sorted from smallest (left) to largest (right) mean inhibition zone diameter. Statistical comparison of inhibition zones was performed with 0.1% Hexetidine as a reference.

FIG. 4: Percent survival in the presence of 0.03% H₂O₂ in PBS (simulates oxidative stress conditions relative to starting inoculum (100%)).

FIG. 5: Adhesion of in-house S. salivarius isolates (indicated with AMBR numbers) to respiratory epithelial cells (Calu-3). The adhesion percentage was statistically compared to that of S. oralis 89a of the Rinogermina® probiotic nasal spray. Rinogermina® S. salivarius 24SMB was also included in this assay. Isolates are sorted from lowest to highest median adhesion percentage. **: p<0.01, ****: p<0.0001. p-values adjusted by the holm method.

FIG. 6: Immunostimulatory capacity of S. salivarius AMBR158 compared to other S. salivarius isolates and the model probiotic Lacticaseibacillus rhamnosus GG (LGG). Induction of (A) nuclear factor kappa-light-chain-enhancer of activated B cells (NF-κB) and (B) interferon regulatory factor (IRF) pathways in THP1-Dual™ monocytes, and (C) TLR2/6 receptor activation in HEK-Blue™ hTLR2-TLR6 reporter cells co-cultured with bacteria are depicted. Results per condition are presented as mean±standard deviation. Statistical analysis was performed with One-way ANOVA with Dunnett's multiple comparisons test against the medium condition representing reporter cells as such, and Poly(I:C) and Pam2CSK4 controls were used to induce immune stimulation; *: p<0.05; **: p<0.01; ***: p<0.001; ****: p<0.0001.

FIG. 7: Phylogenetic tree of S. salivarius strains. Light-gray dots and boxes indicate isolates from the present study, whereas black dots indicate S. salivarius strains K12 and M18 which are already commercialized as probiotics. The genome of S. salivarius 24SMB was not publicly available. AMBR158 is highlighted with an asterisk

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

When describing the compounds of the invention, the terms used are to be construed in accordance with the following definitions, unless a context dictates otherwise.

As used in the specification and the appended claims, the singular forms “a”, “an”, and “the” include plural referents unless the context clearly dictates otherwise. By way of example, “a compound” means one compound or more than one compound.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

The present invention is based on the identification of a novel isolated strain of the Streptococcus salivarius (S. salivarius) species. Said strain has been deposited with the Belgian Co-ordinated Collection of Micro-Organisms (BCCM) on May 28, 2020 with accession number LMG P-31813. In some parts of the application, said strain is also referred to as AMBR158.

As also further detailed in the example, the inventors of the present application found that the LMG P-31813 strain showed a superior capacity to inhibit the growth of several respiratory infection-related micro-organisms, including Haemophilus influenzae, Moraxella catarrhalis, Streptococcus pneumoniae, Streptococcus pyogenes, Staphylococcus aureus, Alloiococcus otitis and Corynebacterium otitidis. As such the bacterial strain shows potential as a probiotic for prevention and/or treatment of respiratory infections such as otitis media.

In accordance with an embodiment of the present invention, the bacteria of the isolated bacterial strain can be provided in suspension, freeze-dried, spray-dried in live or postbiotic form, provided that the active components are not inactivated In the context of the present invention, by means of the term “live form”, reference is made to a form wherein the bacteria are alive. In the context of the present invention, by means of the term “postbiotic form”, reference is made to a form wherein the bacteria are not alive, such as in the case of inanimate applications or tyndalized versions of the bacteria.

The present invention also provides the secondary metabolites of the isolated bacterial strain LMG P-31813. The S. salivarius species is a well-known producer of secondary metabolites with bacteriostatic or bactericidal activity against a range of other bacterial species (8), but the isolated bacterial strain LMG P-31813 showed the unique feature of having two bacteriocin loci and one lassopeptide locus.

Also a composition comprising the isolated bacterial strain or a secondary metabolite thereof is provided. The preparation of the compositions of the invention can be implemented by freeze-drying or spray-drying of bacterial cultures or their secondary metabolites, mixing the dried bacteria or secondary metabolite(s) both in suspension with water or with further suitable excipients and optionally with addition of further active principles.

As used herein, a “composition”, refers to any mixture of two or more products or compounds (e.g. agents, modulators, regulators, etc.). It can be a solution, a suspension, liquid, powder or a paste, aqueous or non-aqueous formulations or any combination thereof. In the context of the present invention, the compositions are preferably pharmaceutical compositions, comprising one or more pharmaceutically excipients, carriers, or diluents, such as suitable sugars, copolymers PEG/PPG, or cryoprotectants.

In one embodiment, said composition comprises one or more pharmaceutically acceptable excipients, aromatizing agents or carriers. Examples of excipients that can be selected in such compositions are rubber, xanthan, carboxymethyl cellulose, silicone, Vaseline, white soft paraffin, magnesium stearate, maltodextrin, mannitol, starch, glucose, trehalose, glycerine, propylene glycol, lactose, and similar.

The compositions may also comprise aromatizing agents; such as thyme or any extracts thereof.

In accordance with an embodiment of the present invention, the carriers provide an improvement of the bioavailability, the stability and/or the endurance of the microorganism.

The compositions may further comprise one or more carriers in order to improve the bioavailability, the stability and the endurance of the micro-organism or its secondary metabolites. The carrier may also improve the adhesion of the bacterial strain or its secondary metabolites on the mucosal surface, such as for example exopolysaccharides produced by S. salivarius or lactobacilli. Further, the carrier may be a heat-sensitive polymer able to increase the viscosity and thus the adhesiveness by increasing the temperature or Gantrex for example. In another embodiment, the carrier can be hydroxypropyl methylcellulose (HPMC).

In accordance with an embodiment of the present invention, the composition comprises an amount of bacteria that is preferably in the range between 10³ to 10¹¹ CFU for each gram of the composition.

In a further aspect, the present invention relates to the isolated bacterial strain, the secondary metabolites or the composition according to the invention for use as a medicament in human or veterinary medicine. In particular, the isolated bacterial strain, the secondary metabolite or the composition according to the present invention are for use in the treatment and/or prevention of infections and/or inflammatory diseases. Said infections and/or inflammatory diseases can be selected from, but not limited to, upper respiratory tract infections; ear, nose, and throat (ENT) infections; oral cavity infections; caries; sinusitis; nasal polyposis; acute otitis media; recurrent otitis media; otitis media with effusion; chronic suppurative otitis media; mastoiditis; halitosis; respiratory infections associated with cystic fibrosis, Covid-19. In an even more preferred embodiment, the isolated bacterial strain, the secondary metabolite or the composition of the present invention is for use in the treatment of otitis media with effusion. In another embodiment, the isolated bacterial strain, the secondary metabolite or the composition according to the present invention are for use in the treatment and/or prevention of immune-related diseases, such as hay fever, allergic rhinitis, allergic sinusitis, asthma, Covid-19 and the like.

In another embodiment, the isolated bacterial strain, the secondary metabolites or the composition according to the present invention are for use in immunomodulation; in particular wherein the isolated bacterial strain, the secondary metabolite or the composition is used as an adjuvant to promote an immune response during vaccination. For example, the inventors of the present application have shown that co-culturing the isolated bacterial strain AMBR158 induces nuclear factor kappa-light-chain-enhancer of activated B cells (NF-κB) and interferon regulatory factor (IRF) pathways in THP1-Dual™ monocytes, in addition to TLR2/6 receptor activation in HEK-Blue™ hTLR2-TLR6 reporter cells. In an even more preferred aspect, vaccination is selected from vaccination against respiratory infections, such as for example respiratory infections caused by coronaviruses, for example SARS-CoV virus or SARS-CoV-2 virus.

The terms “treatment”, “treating”, “treat” and the like refer to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” covers any treatment of a disease in a mammal, in particular a human, and includes: (a) preventing the disease or symptom from occurring in a subject which may be predisposed to the disease or symptom but has not yet been diagnosed as having it; (b) inhibiting the disease symptoms, i.e. arresting its development; or (c) relieving the disease symptoms, i.e. causing regression of the disease or symptom. In the context of the present invention, the terms “prevention” and the like refer to preventing a disease or conditions from happening. In the context of the present invention, the term “immunomodulation” refers to the process of altering an immune response to a desired level and/or direction.

In a further aspect, the present invention relates to the use of the isolated bacterial strain, the secondary metabolites, or the composition according to the present invention in the personal hygiene industry, cleaning industry, air purification, or the production of personal care/consumer products. Personal hygiene industry includes the production of personal care/consumer products for personal hygiene, such as for example tissues, protective masks or sprays; even more in particular in the production of tissues, protective masks or sprays for the treatment and/or prevention of respiratory infections. In a specific embodiment, the use of the isolated bacterial strain, the secondary metabolite or the composition according to the present invention is provided in the oral hygiene industry; in particular in the production of oral hygiene personal care products, such as tooth paste, tooth brushes, or mouth wash solutions.

In another aspect, the present invention provides the use of the isolated bacterial strain, the secondary metabolites or the composition according to the present invention as a probiotic; in particular as a probiotic in the food industry; even more preferred as a probiotic in the production of dairy and non-dairy fermentation products, dietary supplements, dietary food additives and/or nutraceuticals. Said food industry can thus encompass fermented food products (dairy-based, worth, soy). Said food industry can also include the bioreactors and processing environments used in the production of food products, wherein the bacterial strain, the secondary metabolites or the composition of the present invention can be added to the food products during production.

The isolated bacterial strain, the secondary metabolites or composition according to the present invention can be in any form suitable to be administered topically, orally or through the respiratory tract. Further, the bacterial strain, the secondary metabolites or composition according to the present invention can be in a pharmaceutical form selected from, but not limited to, a spray, cream, a lotion, a gel, an ointment, a solution, a suspension, an emulsion, a capsule, a tablet, a powder, a granule, drops, inhaler, tooth paste, mouth wash. Finally, the bacterial strain, the secondary metabolites or composition according to the present invention can be formulated in such a manner that can be administered through the respiratory tract by a nebulizer, with or without propellants.

As used herein, the term “biological sample”, or eventually simply “sample” can encompass a variety of fluid samples, including blood and other liquid samples of biological origin, or tissue samples, or mixed fluid-cell or mixed fluid-tissue samples, obtained from an organism that may be used in a diagnostic or monitoring assay. The term specifically encompasses a clinical fluid or tissue sample, and further includes cell supernatants, cell lysates, serum, plasma, urine, amniotic fluid, biological fluids, tissue biopsies, lavages, aspirates, sputum or mucus. The term also encompasses samples that have been manipulated in any way after procurement, such as treatment with reagents, solubilization, or enrichment for certain components.

### EXAMPLES

### Case-Control Microbiome Study of Chronic Otitis Media with Effusion (OME) in Children Points at Streptococcus salivarius as Pathobiont-Inhibiting Species

### Materials and Methods

### Case-Control Microbiome Study to Identify of Health- and OME-Associated Bacteria

### Study Design

Ethical approval was obtained from the ethical committee of the Antwerp University Hospital for inclusion of OME and cochlear implant patients (B300201731724; clinicaltrials.gov identifier NCT03109496), for the NPcarriage study (B300201526558) and for 16S sequencing of a subset of samples thereof (B300201940224), and informed consent was obtained from a parent or legal guardian before sampling. OME patients were recruited from a group of children aged 1 to 10 years, receiving unilateral or bilateral tympanostomy tubes with or without concurrent adenoidectomy to relieve symptoms of persistent (≥3 months) OME. One control group consisted of microbiologically healthy cochlear implant recipients aged 1 to 45 years and a second control group consisted of children aged 6-30 months healthy enough to attend day care (originally sampled for the NPcarriage study). Exclusion criteria were comorbidities affecting the URT anatomy, immune system or mucociliary system, acute or chronic URT infection, and use of antibiotics or steroids up to one week before surgery Swabs of the anterior nares, nasopharynx, and ear canal, middle ear effusion aspirate, and, in case of simultaneous adenoidectomy, both tissue and swabs of the adenoids were collected from OME patients. Cochlear implant controls provided anterior nare and nasopharynx swabs and a middle ear wash, while day care children provided a nasopharynx swab.

16S rRNA Gene V4 MiSeq Sequencing

DNA of samples from cochlear implant and OME patients was extracted with the QIAamp PowerFecal DNA Kit and quantified with the Qubit 3.0 Fluorometer (Thermo Fisher Scientific). NPcarriage study samples were received as NucliSENS® easyMAG® (BIOMERIEUX) extracted DNA. For all sample, the V4 region of the bacterial 16S rRNA gene was amplified using the barcoded primers 515F (5′-TATGGTAATTGTGTGCCAGCMGCCGCGGTAA-3′; SEQ ID No: 1) and 806R (5′-AGTCAGTCAGCCGGACTACHVGGGTWTCTAAT-3′; SEQ ID No: 2) whereby each sample within a run was indexed with a unique combination of a forward and reverse primer-barcode (9). The PCR mix consisted of 200 μM deoxyribose nucleoside triphosphates (dNTPs), 3% dimethyl sulfoxide (DMSO), 1× Phusion HF Buffer, 0.4 units of Phusion™ High-Fidelity DNA Polymerase (Thermo Fisher Scientific), 0.5 μM each of the forward and the reverse primer and a maximum of 50 ng or 5 μL of DNA extract. This mixture was supplemented with PCR-grade water to a final volume of 20 μL. The amplification conditions were 30 cycles of 20 seconds denaturation at 95° C., followed by 20 seconds annealing at 55° C. and 1 minute elongation at 72° C. Cycling was preceded by an initial denaturation step of 2 minutes and concluded with a final elongation step of 10 minutes. The amplicons were purified using Agentcourt AMPure XP (Beckman Coulter, A63881) according to the protocol, with elution in 40 μL PCR-grade water. This was followed by DNA quantification with the Qubit Fluorometer 3.0 on 2 μL amplicon and same-day equimolar pooling. The pooled amplicons (˜282 bp) were then separated from other DNA fragments through gel-electrophoresis (50 min at 60 V in 0.8% agarose) and extracted from the gel with the NucleoSpin® Gel and PCR Clean-up kit (Macherey-Nagel) according to the protocol with elution in a final volume of 15 μL. The library was quantified, and diluted to 2 nM, 5 μL of which were loaded on the MiSeq Desktop Sequencer (Illumina).

### Data Analysis

After sequencing of the V4 region of the 16S rRNA gene, trimming, error correction, chimera removal and classification of paired reads against the EzBioCloud 16S database version of 19.01.2018 were all performed in DADA2 version 1.6.0. This workflow resulted in an ASV (amplicon sequence variant) table with a single nucleotide difference resolution. Sequenced extraction and PCR controls served as indicators of background contamination. For contaminant filtering and data visualization, packages included in tidyverse 1.2.1 and the tidyamplicons package (https://github.com/SWittouck/tidyamplicons) were used. ASVs of interest were further classified using the online EzBioCloud 16S-based ID web-app (Update 2020.05.13).

The differential abundance of ASVs between the nasopharynx of OME patients and of controls was calculated using the ANCOM (Analysis of Composition of Microbiomes) R tool (version 1.1.2). PERMANOVA (vegan version 2.2.5) was used to determine the effect of metadata on microbiome composition and to compare the same anatomic location between cases and controls. The mean age of cases and controls was compared using Student's t-test (ggpubr version 0.2.5). The 16S rRNA gene sequencing data generated for this study were deposited in the European Nucleotide Archive under accession number PRJEB33591.

Characterization of Isolates from the Healthy URT

### Isolation of Lactic Acid Bacteria

Samples from cochlear implant controls were plated out on three different agar media (Table 1). From each plate, one colony per morphology was selected. Bacteria were identified by Sanger sequencing of the 16S rRNA gene (primers 27F (5′-AGAGTTTGATCMTGGCTCAG-3′; SEQ ID No: 3) and 1492R (5′-GGTTACCTTGTTACGACTT-3′; SEQ ID No: 4).

TABLE 1
Cultivation conditions

| | | |
|---|---|---|
| Samples or Target Bacteria | Growth Medium | Atmosphere |

Plating out of healthy control samples (cochlear implant group)

| | | |
|---|---|---|
| Lactobacilli | MRS | 5% CO₂ |
| Lactococci | M17 | 5% CO₂ |
| Dolosigranulum pigrum | BHI + 5% (v/v) Tween 80 | 5% CO₂ |

Respiratory and middle ear pathogens

| | | |
|---|---|---|
| Alloiococcus otitidis | BHI + 5% horse blood | Aerobic (shaking) |
| Corynebacterium otitidis | BHI + 0.5% Tween 80 | 5% CO₂ |
| Haemophilus influenzae | MH + 0.5% yeast extract + | 5% CO₂ |
| | 15 mg NAD + 15 mg Hemin | |
| Moraxella catarrhalis | MH | |
| Staphylococcus aureus | MH or MRS | Aerobic |
| Streptococcus pneumoniae | TH | 5% CO₂ |
| Streptococcus pyogenes | TH + 0.2% (w/v) yeast extract | |

(Potential) probiotics

| | | |
|---|---|---|
| Streptococcus salivarius | TH | Aerobic or 5% CO₂ |
| Streptococcus oralis | TH | Aerobic or 5% CO₂ |

Note:
All bacteria were incubated at 37° C. BHI, Brain Heart Infusion; MH, Mueller Hinton; MRS, De Man, Rogosa and Sharpe; TH, Todd Hewitt; NAD, Nicotinamide adenine dinucleotide.

### Antimicrobial Screening Assays

The ability of isolates to inhibit the growth of URT and middle ear pathogens was tested (1) by overlaying 48 h 2 μL spots of isolate with pathogen-containing soft agar (Spot Assay), and (2) by inoculating 30 μL spent filter-sterilized culture supernatant into wells punched into pathogen-containing agar (Radial Diffusion Assay) [17]. 0.1% Hexetidine (Hextril®, Johnson & Johnson) and Todd Hewitt (TH) broth served as positive and negative controls, respectively. For some repetitions, the pH of the TH broth was reduced to 5. Growth conditions are summarized in Table 1.

### Antibiotic Susceptibility Assay

Minimal inhibitory concentrations of antibiotics (ampicillin, vancomycin, gentamicin, streptomycin, erythromycin, clindamycin, tetracycline and chloramphenicol) were determined using a broth microdilution assay with two-fold serial dilutions between 0.5 μg/mL and 128 μg/mL with evaluation of presence/absence of growth after 24 h of incubation. Cut-off values for Streptococcus thermophilus were used based on the guidelines of the European Food Safety Authority (EFSA).

### Adherence Assay to Airway Epithelial Cells

The human airway epithelial cell line Calu-3 ATCC® HTB-55TM (purchased from ATCC, Molsheim Cedex, France) was cultured in 75 cm² flasks containing 20 mL Minimum Essential Medium (MEM) (Life Technologies, Erembodegem, Belgium) supplemented with heat inactivated fetal bovine serum (Thermo Fischer, Asse, Belgium) and penicillin-streptomycin (100 U/mL) (Life Technologies) and maintained in a humidified 5% CO₂ incubator at 37° C. The culture medium was changed every 3-4 days and the cells were passaged weekly at a 1:2 split ratio using a 0.25% trypsin-EDTA solution (Life Technologies).

To test the ability of bacterial isolates to adhere to human respiratory epithelium, 2*10⁸ colony forming units (CFU) of bacteria were added to fully grown Calu-3 cultures seeded at a density of 3*10⁵ cells/cm². After 1 h incubation at 37° C. and 5% CO₂, unattached bacteria were removed by washing the cells once with PBS. This was followed by trypsinization for 15 min at 37° C. and 5% CO₂ to detach the Calu-3 cells and adherend bacteria. Triplicate ten-fold serial dilutions in PBS (10⁻² to 10⁻⁷) of left-over cell-suspension (before) and of adherend cells were plated on TH agar and incubated overnight at 37° C. and 5% CO₂. Colonies were counted after incubation and the adhesion percentage was calculated by comparing the number of CFU added to the Calu-3 cells to the number of CFU retrieved after the adhesion experiment

### Oxidative Stress Resistance Testing

### Hydrogen Peroxide Assay

2×10⁸ CFU of an overnight culture were pelleted for 10 minutes at 1400 g, washed twice with PBS using the same centrifugation settings, and dissolved in 2 mL PBS. Two hundred microlitres were used to create 10-fold serial dilutions in PBS in triplicate, of which 10 μL were spotted on appropriate agar medium. Then 1350 μL were combined with 0.3% (0.979 M) hydrogen peroxide (C_{Final}=0.03% w/w=0.0979 M) and incubated at 37° C. under shaking. Aliquots were removed after 20, 40 and 60 minutes, serially diluted, and spotted on appropriate agar. Colonies were counted the next morning (Streptococci) or after 1.5 days (Lactobacilli). L. casei AMBR2 and S. salivarius 24SMB and S. oralis 89a (isolated from the probiotic nasal spray Rinogermina®, DMG Italia) served as controls.

### Induction of NF-κB and IRF Pathways in Human Monocytes

The THP1-Dual™ NF-κB-SEAP and IRF-Lucia luciferase human reporter monocytes were cultured according to the manufacturer's (InvivoGen) instructions. For the NF-κB and IRF pathway induction assessment, THP1-Dual™ cells were seeded in 96-well plates at a concentration of 10⁵ cells/well and combined with 10⁶ CFU/well of UV-inactivated bacteria. The Poly(I:C) control with lipofectamine 2000 at 25 μg/mL was used to induce IRF. After 24 h of co-incubation at 37° C. and 5% CO₂, the NF-κB induction was assessed via monitoring SEAP activity after addition of a p-Nitrophenyl Phosphate (pNPP) solution by measuring the optical density at 405 nm in each well of the 96-well plate. IRF induction was assessed by monitoring luciferase activity in each well of the 96-well plate. Both read-outs were performed using the Synergy™ HTX Multi-Mode Microplate Reader.

### Induction of TLR2/6 in Human Cells

The HEK-Blue™ hTLR2-TLR6 reporter cells were cultured according to the manufacturer's (InvivoGen) instructions. For TLR2/6 activation assessment, HEK-Blue™ hTLR2-TLR6 cells were seeded in 96-well plates at a concentration of 2×10⁵ cells/well and incubated for 24 h at 37° C. and 5% CO₂. Subsequently, 10⁶ CFU/well of UV-inactivated bacteria were added to the HEK-Blue™ hTLR2-TLR6 cells. The Pam2CSK4 control at 25 ng/mL was used to induce TLR2/6. After 24 h of co-incubation at 37° C. and 5% CO₂, TLR2/6 activation was assessed via monitoring SEAP activity after addition of a p-Nitrophenyl Phosphate (pNPP) solution by measuring the optical density at 405 nm in each well of the 96-well plate using the Synergy™ HTX Multi-Mode Microplate Reader.

### Phage Induction Assay

S. salivarius overnight cultures were diluted 1 in 50 in TH broth and incubated for 30 minutes at 37° C., before Mitomycin C was added to a final concentration of either 0, 0.1 or 0.2 μg/mL. The culture density was measured hourly at 600 nm (OD600) for 15 hours. Then the cells were pelleted for 10 minutes at 1400 g, 4° C. and the supernatant was neutralized to a pH of 7 to 7.2 using 0.1 M NaOH, followed by filter-sterilization. Square petri dishes with 45 mL base TH agar and 15 mL top TH soft agar (0.65%) containing 300 μL S. salivarius overnight culture were prepared and 10 μL phage-induction supernatants were spotted on the surface. After drying, the plates were incubated at 37° C. followed by evaluation for clearing zones due to phage lysis.

### Bacterial Whole Genome Sequencing: DNA Extraction and Data Analysis

Bacterial DNA was extracted for whole genome sequencing (WGS) as follows: 2×1.5 mL bacterial overnight culture were incubated for 1 h at 37° C., each in the presence of 1.5 μl (100 mg/ml) ampicillin. Next, the bacteria were pelleted (12000×g for 3 min), washed three times with 1 mL NaCl-EDTA, followed by resuspension of both pellets in a single volume of 100 μl NaCl-EDTA, to which 100 μl lysozyme (10 mg/mL in NaCl-EDTA) and 1 μl RNAse (20 mg/mL) were subsequently added. After 1 h incubation at 37° C. under shaking, 229 pl NaCl-EDTA, 50 μl SDS (10%) and 20 μl Proteinase K (20 mg/mL) were added, followed by incubation at 55° C. for 1 hour. Next, proteins were precipitated with 200 μl fridge-cold protein precipitation solution consisting of 6 ml of 5M potassium acetate, 1.15 ml glacial acetic acid and 2.85 ml distilled water, followed by 5 min incubation on ice and double centrifugation at 12000×g and 4° C. for 3 min with transfer of the supernatant after each centrifugation step. Then, the DNA was precipitated with 600 μl ice-cold isopropanol, pelleted at 12000×g at 4° C. for 3 min and washed once with 70% ethanol. Finally, the supernatant was discarded, the DNA pellet was air-dried and dissolved in 100 μl H₂O through 5 min incubation at 55° C.

The DNA was sequenced on an Illumina MiSeq platform and the resulting reads were assembled de novo with SPAdes-based Shovill (https://github.com/tseemann/shovill), followed by quality control with checkM and annotation with Prokka. Assembled contigs were screened for the presence of transferable antibiotic resistance genes against the ResFinder 3.2 database, and for virulence factors against the Virulence Factor Database (VFDB) using ABRicate (https://github.com/tseemann/abricate). Secondary metabolites were identified by antiSMASH 5.0 and BAGEL4. Genes of interest were further characterized using NCBI-BLAST.

Phylogenetic tree of S. salivarius

NCBI assembly accession numbers of genomes classified as Streptococcus salivarius or Streptococcus sp001556435 with a maximum contamination and minimum completeness rate of 5% and 98%, respectively, were retrieved from the Genome Taxonomy Database (GTDB). The assembled genomes were downloaded, and genes were predicted and translated with Prodigal. Next, single-copy core genes were identified based on a subset of genomes using progenomics, followed by calculation of the genes' occurrence across all genomes, which led to a selection of >1100 genes present in a single copy in ≥99% of genomes. These genes were aligned, the alignments were concatenated, and columns where >2% of sequenced had a gap were discarded. Finally, the phylogenetic tree was built using IQ-TREE with a GTR+G substitution model) and visualized in R with midpoint rooting using the phytools package.

### Results

### Study Population and Sample Characteristics

Samples were collected from 70 OME patients, 12 cochlear implant recipients and 41 day-care children. Of 523 samples sequenced, 443 were retained after quality filtering, with library sizes ranging from 2500 to 784460, with 36332±49509 reads (mean±standard deviation). There was no significant age difference between the OME group and the combined control group (4.38±2.42 years vs 4.56±7.21 years, p=0.12). In the cochlear implant group, 42% of participants were female, comparable to 41% of both the OME patients and day care controls. Sixty-six OME patients received tympanostomy tubes in both ears and 28 patients underwent adenoidectomy. Gender had no significant effect on microbiome composition and age only influenced the anterior nare microbiome (p=0.002 in the OME group and p=0.043 in the cochlear implant group). Neither laterality (uni-vs bilateral OME) nor adenoidectomy significantly influenced the microbiome composition of any of the sampled locations.

### Pathobiont Characteristics of the Middle Ear During OME

Of 97 OME effusion samples 80% were dominated (≥50% relative abundance) by a single ASV (Table 2). In only 33% of effusions, the dominant ASV belonged to one of the classic otopathogen genera Haemophilus, Moraxella and Streptococcus, but these genera showed a high prevalence, with detection in 75%, 53% and 56% of middle ear samples, respectively.

Other dominant ASVs were Alloiococcus 1 (39%), Turicella 1 (4%), Staphylococcus 1 (3%) and Corynebacterium 1 (1%). To explore body site continuity, we plotted the similarity (1—Bray Curtis dissimilarity) between the middle ear effusion and nasopharynx microbiome against the similarity between the middle ear effusion and the side-matched ear canal microbiome. All samples dominated by Alloiococcus, Turicella, Staphylococcus or Corynebacterium were more similar to the ear canal (similarity score≥0.574) than to the nasopharynx (similarity score≤0.254). These taxa were also frequently dominant in the ear canal of all patients (data not shown), indicating that these taxa probably originated from the ear canal.

TABLE 2
Prevalence of ASVs dominant (≥50% relative abundance) in at least
one middle ear effusion

| | | Middle Ear | | Ear Canal | |
|---|---|---|---|---|---|
| | | Prevalence | Dominance¹ | Prevalence | Dominance |
| | | (%) | (%) | (%) | (%) |
| Alloiococcus 1 | A. otitidis | 87.6 | 39.2 | 90.2 | 75.5 |
| Haemophilus 1 | H. influenzae | 56.7 | 14.4 | 46.1 | 0 |
| Haemophilus 2 | H. aegyptius | 42.3 | 10.3 | 22.5 | 0 |
| Streptococcus 3 | S. pyogenes | 4.1 | 1.0 | 2.9 | 0 |
| Streptococcus 1 | S. pneumoniae/ | 14.4 | 2.1 | 4.9 | 0 |
| | pseudopneumoniae | | | | |
| Corynebacterium 1 | C. pseudodiphtheriticum/ | 8.2 | 1.0 | 13.7 | 1.0 |
| | propinquum | | | | |
| Haemophilus 3 | H. quentini/influenzae | 10.3 | 1.0 | 1.0 | 0 |
| Moraxella 1 | M. catarrhalis/ | 47.4 | 4.1 | 43.1 | 0 |
| | nonliquefaciens | | | | |
| Staphylococcus 1 | Not resolved to species level | 51.5 | 3.1 | 69.6 | 4.9 |
| Turicella 1 | Corynebacterium otitidis | 71.1 | 4.1 | 81.4 | 7.8 |

¹Percentage of samples with a relative abundance ≥50%

### The Microbiome of the Healthy Middle Ear Cavity

Twelve middle ear rinses collected from microbiologically healthy cochlear implant recipients were sequenced to identify bacteria associated with middle ear health. However, only four of these had at least twice the number of reads compared to the largest negative control after removing obvious contaminants (data not shown) and were retained after quality filtering. In addition, of the 107 ASVs detected in the remaining four samples, only seven were present in multiple samples (data not shown), and of these, only Streptococcus 1 and Corynebacterium 1 were absent from the negative controls. ASVs detected in the negative controls but expected to be found in the respiratory tract and ear environment were not removed from the dataset. While their effect is minor in higher biomass samples, care needs to be taking in interpreting their presence in samples of extremely low biomass such as the healthy middle ear. Attempts to sequence the microbiome of the healthy middle ear were therefore considered unsuccessful, indicating that very few or even no bacteria were present, in accordance with a recent study which argued that bacterial signals detected in this body site under healthy conditions are likely due to contamination.

### Comparison of the Nasopharynx Microbiome in Health and During Chronic OME

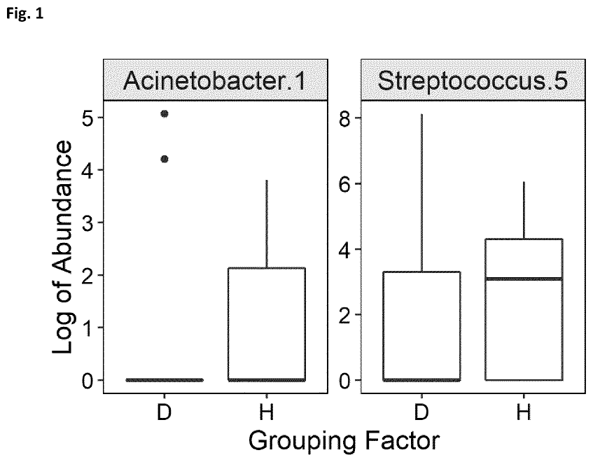

To be able to compare the URT microbiome of OME patients with healthy controls and to identify health-associated bacteria, we decided to focus on the nasopharynx instead of the middle ear. The nasopharynx is the natural habitat of the classic middle ear pathobionts, making it a suitable and accessible location for probiotics targeting middle ear health. The nasopharynx microbiome differed significantly between patients and controls (p=0.014), a difference which was more pronounced than that observed between the two control groups (p=0.049). A total of 134 taxa were shared between both control groups and the OME group (data not shown). Differential abundance analysis (ANCOM) with stringent correction for multiple testing using the combined control dataset identified Acinetobacter 1 (A. lwoffii or A. pseudolwoffi) and Streptococcus 5 (S. salivarius, S. thermophilus or S. vestibularis) as health-associated (FIG. 1). The latter extends the results of a recent study, which detected a taxon related to S. thermophilus to be significantly more abundant in the anterior nare of healthy controls than of chronic OME patients (10)

Antimicrobial Activity of S. salivarius against URT Pathobionts

We then aimed to characterize the potential beneficial properties of bacteria isolated from healthy controls in more detail, especially with their potential to control middle ear pathobionts. Our culturomics approach started with the isolation of 142 bacterial isolates belonging to 11 different genera (data not shown). Streptococcus spp. were most frequently isolated (n=66), especially from the nasopharynx, while A. llwoffii or A. pseudolwoffii isolates were not obtained, likely due to their low relative abundance (0.1%) in cochlear implant controls. All Streptococcus isolates from healthy children belonged to either the mitis group (n=28), the salivarius group (n=32), or the sanguinis group (n=5).

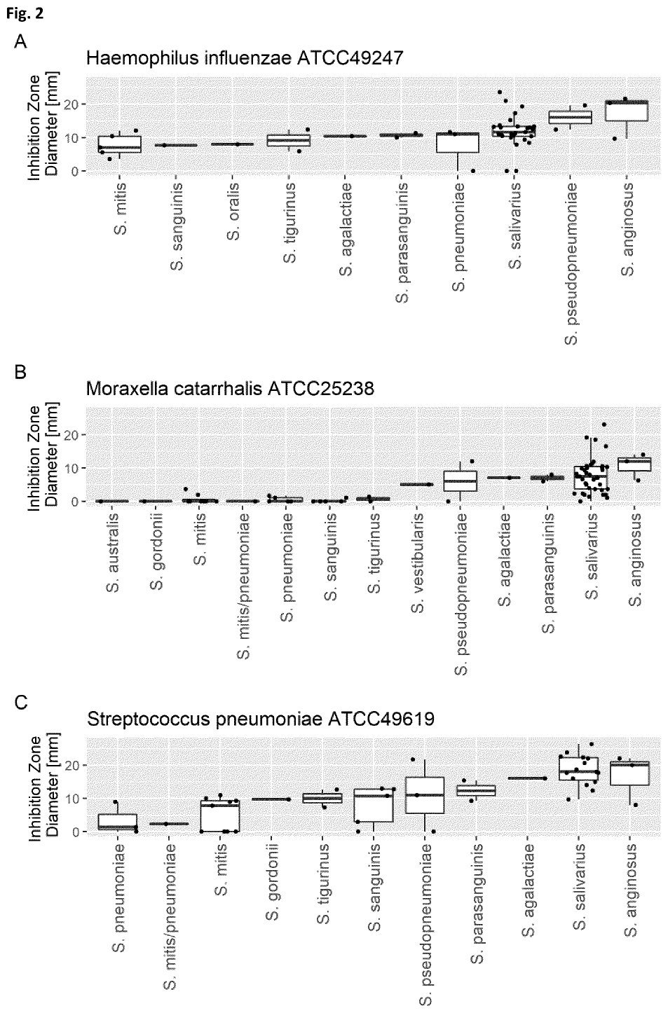

Seventy-eight Streptococci (53 from this study and 25 isolated from healthy adults) were screened for their antimicrobial activity. All tested species could inhibit the growth of H. influenzae, with S. anginosus, S. pseudopneumoniae and S. salivarius showing the largest inhibition zones (FIG. 2A). S. anginosus and S. salivarius could also inhibit M. catarrhalis, but this effect was strain-dependent (FIG. 2B). S. salivarius and S. vestibularis were most effective against S. pneumoniae, with S. anginosus in third place (FIG. 2C).

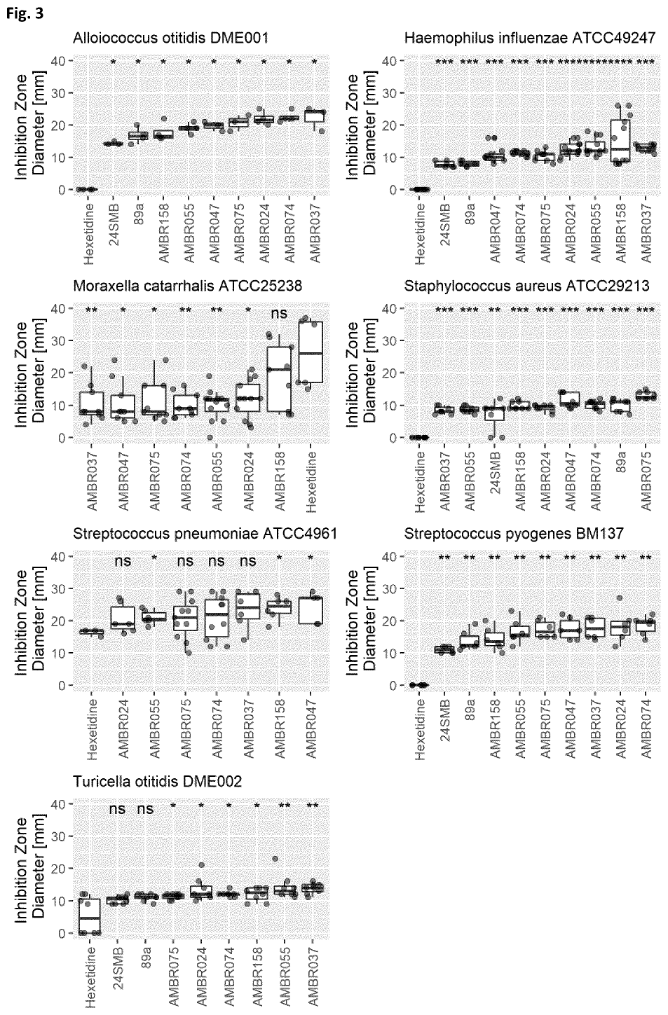

Seven S. salivarius isolates (AMBRO24, AMBRO37, AMBRO47, AMBRO55, AMBRO74, AMBRO75 and AMBR158) were selected for WGS and more detailed in vitro characterization based on their health-association, prevalence, and superior ability to inhibit the growth of the classic middle ear pathobionts. In addition to the tests against H. influenzae, M. catarrhalis and S. pneumoniae, these isolates were also tested against the URT pathobionts Streptococcus pyogenes and Staphylococcus aureus, and the suspected middle ear pathobionts A. otitis and Corynebacterium otitidis (formerly Turicella otitidis) isolated from OME middle ear effusion during this study. S. salivarius 24SMB and S. oralis 89a isolated from the probiotic nasal spray Rinogermina® (DMG ITALIA) were used as references. These isolates could inhibit all tested pathobionts in spot-assays (FIG. 3), whereby AMBR158 was the most effective isolate against the classic otopathogen genera.

Prediction of Secondary Metabolites with Antimicrobial Activity

The genomes of the selected S. salivarius isolates were subsequently screened for loci encoding potentially bacteriostatic or bactericidal secondary metabolites. All isolates harbored a class IIc bacteriocin-like peptide (blp) cassette with different predicted bacteriocins, ABC-transporters and immunity proteins. AMBR074, AMBR075 and AMBR037 additionally encoded a class IId Lactococcin 972 family bacteriocin, including an ABC transporter and an immunity mechanism. A lantipeptide locus related to Streptococcin A M49 (13) and Macedocin (14) was found in the genome of AMBRO74. Lassopeptides (bacteriocins class If) were detected in AMBR024 (undetermined) and AMBR158 (related to Streptomonomicin). In addition to bacteriocins, antiSMASH (15) also predicted a Gramidicin NRPS (nonribosomal peptide synthetase) locus in AMBR024 (Table 3). The specific amino acid sequences of the bacteriocin locus 1 and 2 and the lassopeptide locus of the AMBR158 are represented in Table 4.

### Oxidative Stress Resistance

To survive in the URT, bacteria must be adapted to the oxidative stress. Hydrogen peroxide (H₂O₂) is produced by neutrophils, macrophages and some bacteria and plays a role in host-microbe and microbe-microbe interaction.

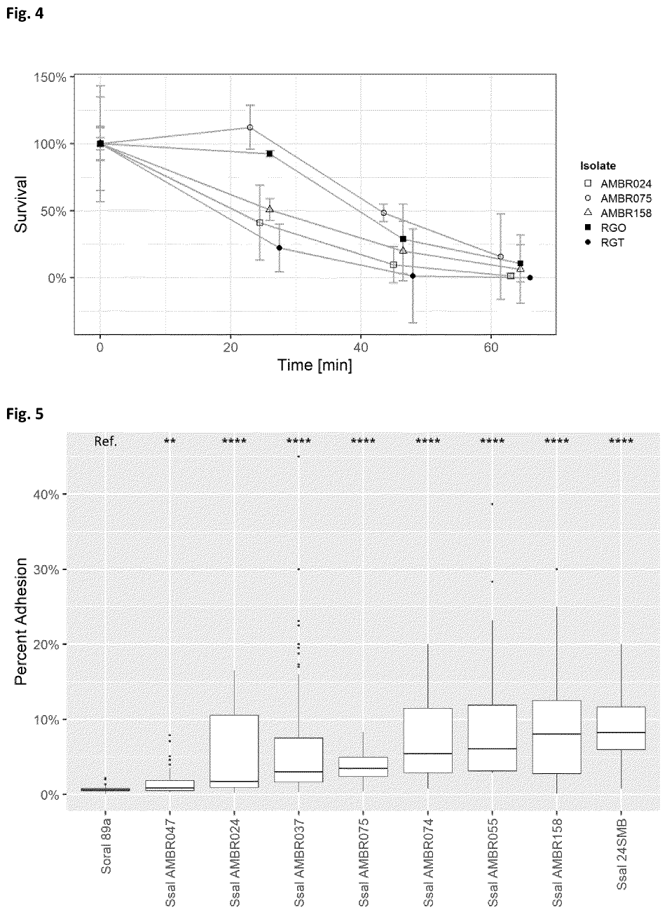

Some respiratory tract bacteria, for example S. pneumoniae, actively produce high levels of H₂O₂ which can inhibit the growth of other bacteria present in the same niche. To phenotypically characterize the capacity of our isolates to survive in H₂O₂ despite the lack of catalase, isolates were exposed to 0.03% H₂O₂ in PBS with plating out before exposure and after 20, 40 and 60 minutes (FIG. 4). All tested isolates showed some resistance to H₂O₂ though their viability decreased over time. S. salivarius isolates were more resistant than S. oralis 89a (RGT)for which no colonies grew after 60 minutes. Exposure to 0.1% H₂O₂ for 90 minutes had killed all isolates in a previous experiment.

### Safety Evaluation

Potential probiotics should not carry antibiotic resistance markers, especially not on mobile elements as these could be transmitted to pathogens, complicating their treatment. We therefore screened the isolates' genomes for antibiotic resistance markers and tested their susceptibility to key antibiotic classes in vitro. S. salivarius AMBR055 and AMBR047 were predicted to harbor the adjacent genes mefA (100% coverage with 96% identity) and mel (100% coverage with 100% identity) which encode efflux pumps for macrolide class antibiotics. Phenotypic testing indicated only resistance for AMBR047 against the macrolide Erythromycin (MIC of 4-16 mg/L with a cut-off of 2 mg/L) and against Chloramphenicol (MIC of 8 mg/L with a cut-off if 4 mg/L). AMBR055 and AMBR047 were excluded from further analysis based on this finding.

As an additional safety check, we also verified that the potential probiotics did not carry genes encoding virulence factors. No virulence genes of concern were observed, although two genes showed a hit with the Virulence Factor database (VFDB) for all isolates: psaA encoding a putative adhesin (with 87.85%-88.92% coverage of and 76.24%-76.96% identity to the pneumococcal surface adhesion gene of S. pneumoniae TIGR4) and hasC encoding UDP-glucose pyrophosphorylase (91.26%-91.85% coverage with 76.94-77.67% identity to the gene of S. pyogenes M1 GAS) probably involved in capsular polysaccharide biosynthesis. These genes were not considered real virulence factors, but rather adaptation factors, reflecting adaptation of S. salivarius to the URT rather than pathogenicity. Sufficient adhesion to host tissue is generally considered as a desired property for most probiotic applications because it increases a probiotic's opportunity to interact with its host and mediates displacement of already adhered bacteria and competitive exclusion of pathogens which bind to the same receptors. For capsular polysaccharides, it is important to check the molecular composition: S. salivarius produces a levan or dextran capsule instead of the known virulence factor hyaluronic acid capsule found in pathogenic S. pyogenes that mimics human connective tissue. Therefore, the hasC gene is not of concern.

Ability of S. salivarius to Adhere to Respiratory Epithelium

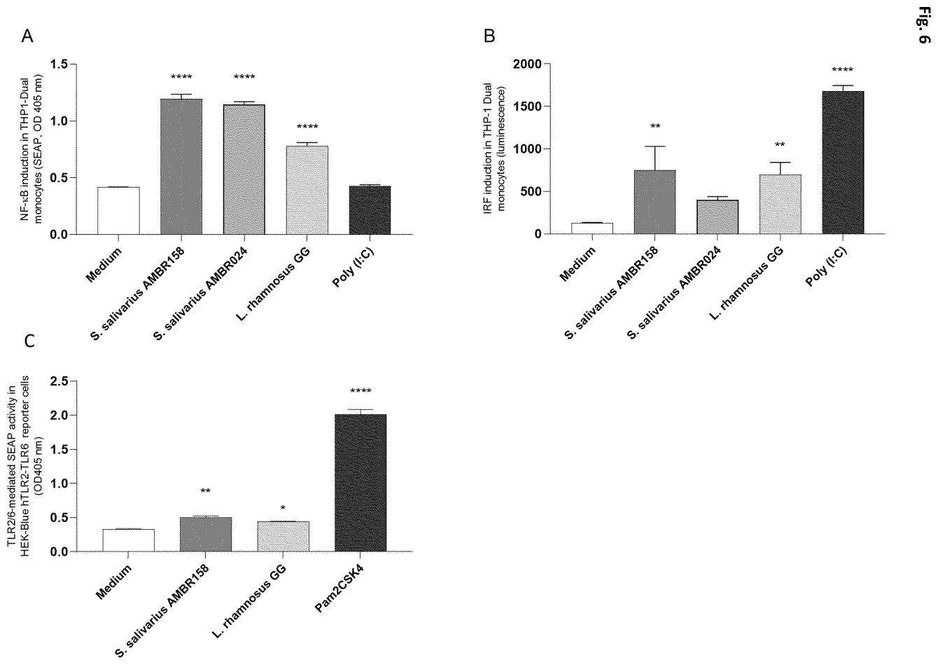

Sufficient adhesion to the host mucosa or epithelial cells is known to increase a probiotic's opportunity to interact with its host and mediates displacement of already adhered bacteria and competitive exclusion of pathogens which bind to the same receptor (11, 12). We therefore phenotypically characterized the interaction of these S. salivarius isolates with the respiratory epithelial cells Calu-3. All isolates could adhere to the cells, with median adhesion values between 1.8% (AMBRO24) and 8.1% (AMBR158) (FIG. 5) in agreement with the fact that they express adhesion factors.

Immunostimulatory Capacity of S. salivarius

Immunostimulatory capacity of S. salivarius AMBR158 compared to other S. salivarius isolates and the model probiotic Lacticaseibacillus rhamnosus GG (LGG) (FIG. 6). Induction of nuclear factor kappa-light-chain-enhancer of activated B cells (NF-κB) and interferon regulatory factor (IRF) pathways in THP1-Dual™ monocytes (resp. Panel A and B), and TLR2/6 receptor activation in HEK-Blue™ hTLR2-TLR6 reporter cells (Panel C) co-cultured with bacteria are depicted.

S. salivarius Phylogeny

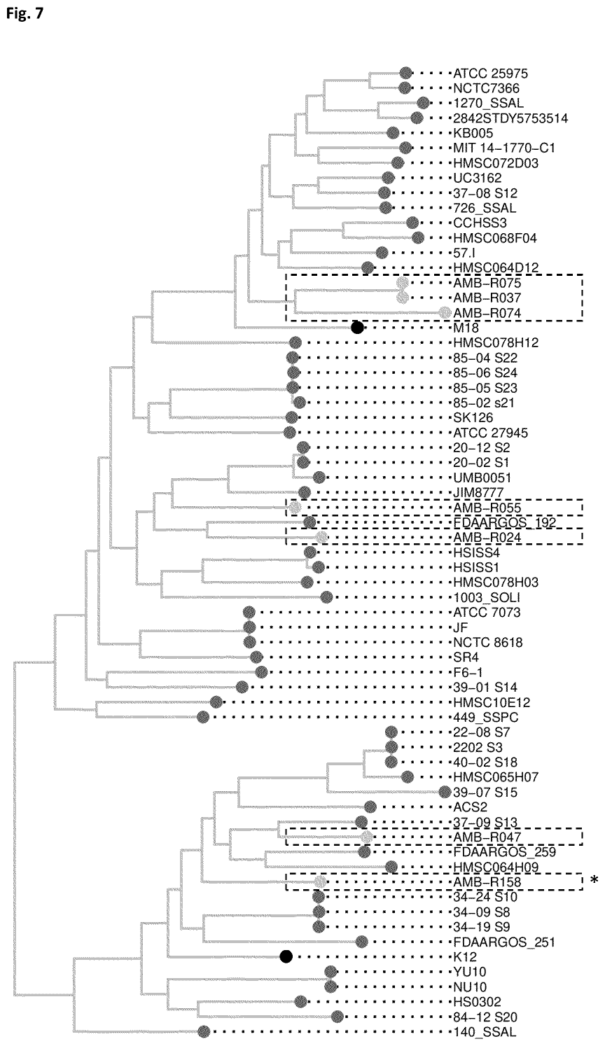

The seven characterized S. salivarius isolates originated from just 3 children and one adult. We therefore explored their relatedness to each other but also to other publicly deposited S. salivarius genomes including the marketed probiotic strains K12 and M18. Strain 24SMB was not available for analysis. A phylogenetic tree is displayed in FIG. 7. Two and three isolates were obtained from subject 1 and subject 6, respectively. AMBR075 and AMBR037 isolated from subject 6 appear to be clonal, a finding which is also supported by an ANI (average nucleotide identity) value of 99.95 between their genomes and the fact that they harbor the same bacteriocin loci and the same incomplete prophage (Streptococcus virus O2105). The S. salivarius genomes appeared to be split into two clades: one Glade contained the isolates from subject 1 (AMBR074, AMBR075, AMBR037) and subject 6 (AMBR024, AMBR055) and the probiotic strain M18. The second Glade contained AMBR047 and AMBR158 together with the probiotic strain K12.

TABLE 3
Secondary metabolite-producing genetic loci detected by BAGEL4 and AntiSMASH.
Names indicated the most closely related bacteriocin type.

| | Class I | | Class II | | | | |
|---|---|---|---|---|---|---|---|
| | Lantipeptides (Ia) | Lassopeptides (If) | IIc (Bip locus) | IId | NRPS | Furan | T3PKS |
| AMBR024 | | Lassopeptide | Mutacin IV | | Gramicidin | | T3PKS |
| AMBR037 | | | Lactococcin | Lactococcin 972 | | | T3PKS |
| AMBR047 | | | Lactococcin | | | | T3PKS |
| AMBR055 | Salivaricin A2/Salivaricin 9 | | Lactocccin & Gassericin | | | AvrD | T3PKS |
| AMBR074 | Streptococcin A | | Lactococcin & Thermophilin A | Lactococcin 972 | | | T3PKS |
| | M49/Macedocin | | | | | | |
| AMBR075 | | | Lactococcin | Lactococcin 972 | | | T3PKS |
| AMBR158 | | Streptomonomicin | Thermophilin A | | | | T3PKS |

TABLE 4
Amino acid sequences of the Bacteriocin locus 1 and 2 and the
Lassopeptide locus of the AMBR158 strain.

BACTERIOCIN LOCUS 1 (related to Thermophilin A)
AMBR158 ctg1_271 (core biosynthetic gene = ComC/Blp family bacteriocin) -
SEQ ID No. 5
MTTQTMNNFETLDLEALANVEGGNRCKDLIFGGALTGAGAGFTGGMAAFGVTAFPGAFVGAHVGAVAGGLACIGAS
F
AMBR158 ctg1_272 (transport-related gene = ABC transporter yheH) - SEQ ID
No. 6
MPNQNQWSVFKRLLTYLKPYKFLTFLALTFLLSTTIIKSIIPLVASYFIDHYLHDMNQAASLILIGYYGLYLLQTL
VQYLGNLFFARVSYSIVRDIRRDAFANMEKLGMSYFDKTPAGSIVSRLTNDTETISEMFSGILSSFISAIFIFVTT
LYTMMILDIRLTGLIVLFLPLIVLLVNLYRKKSVVIIEKTRALLSDINAKLSESIEGIRIIQAFNQEKRLKKEFDA
INEEHLIYASRSMSLDSLFLRPAMSLLKLLGYAVLMAYFGYRGIYLGITAGTMYAFIQYINRLFDPLIEVTQNFST
LQTSMVSAGRVFALIDERTYEPEQRDTDAKVTEGNIRFENVSFSYDGKHQILDNISFSVNKGETIAFVGSTGSGKS
SIINVFMRFYEFQSGRVLLDGVDIRDYSQAELRKNIGLVLQEPFLYHGTIKSNIAMYQDLTDEEIKAAAEFVDANH
FIDKLPEGYDAPVSERGSSFSTGQRQLLAFARTVASQPKILILDEATANIDSETEAIVQNSLAKMRQGRTTIAIAH
RLSTIQDANCIYVLDKGRIIEHGSHEELLALGGTYHKMYSLQAGAMEGA
AMBR ctg1_273 (transport-related gene = ABC transporter yheI) - SEQ ID No. 7
LFFKESIIAKLWWFFKLEKRRYIVGILALSLVSVLNLIPPMVMGRVIDAITSGKLTNQILLLNLVYLLLAAIGMYY
LRYVWRMYILATSYRLGQIMRSRLFEHFTKMSPSFYQKYRTGDLMAHATNDINSLTRLAGGGVMSAVDASITALVT
LITMFFSISWQMTLVAVLPLPFMAFATSRLGRKTHKAFGESQAAFSELNNKVQESVSGIKLTKSFGYQEAELQSFQ
ETNKMTFKKNMHTMKYDSLFDPLVLLFVGSSYVLTLLVGAFMIQAGQITVGNLVTFITYLDMLVWPLMAIGFLFNI
TQRGNVSYQRIENLLEQESDVQDPKHPLPSIENGRLEYAIDRFAFEDEDTLRDIHFTLDKGQTLGLVGQTGSGKTA
LVKLLLREHDVNQGAIYLNGHDIRDYRLSDLRSLMGYVPQDQFLFASSILDNVRFGNPDLAFEKVEEATKLAQVYD
DIKAMPEGFETIIGEKGVSLSGGQKQRLAMSRAMILDPDILILDDSLSAVDAKTEHAIIDNLKHTRKDKTTIITAH
RLSAVVHADLVLVMQNGRIIERGRHEDLLAQGGWYAKTYESQQLEMEGGEVDA
LASSOPEPTIDE LOCUS (Related to Streptomycin)
AMBR158 ctg2_139 (regulatory gene = sensor histidine kinase cssS) - SEQ ID
No. 8
VEKSLTFNQLVKQTYFKILRQFLINLFLFYLLPMLLTAFTNITEKNAGAFTIYFSTASWIYISLILLFSITKNMRN
LLEKIKQETIIVYNQSLLGDTNENNSAKLTLIELIETRQKIQEMQATIKKIIQSEKQQKQELMFQVSAAAHDLKTP
LTVIQGNAQFLQSMDIPGNIEQCLEDIELASQQLNLYFNQLINYSKTFYNDTSNWESLSSDYLFELFEQEIKLITD
KKADIQFSNNLPTSIYLTLNLNLFLRATLNIINNAIDHSQSSYPTIKVNYKFIDNQFYISIWNSDSSFTENLLEKY
GLLFYQDKQATNSEQRSNFGIGLAFVKRVAKIHGGQVNLSNCNNGALVTICIPV
AMBR158 ctg2_140 (regulatory gene = response regulator transcription factor
arLR_1) - SEQ ID No. 9
MKYKILVIDDDKSILRLVRNVLESKNFVVDTLTSIPDIDICHFIGYDLIILDIMMPHSGIEICRYIREFISVPIIF
LTAKNLEEDVVCGIQSGADDYITKPFSIPELVARVQMHIRREVRNNNSNEILQFGPITINKIKEELRVADVHVPTT
KREFQLILLLSSNPNKIFSNEELFDYLYPHDSETQGRSITEYVYQVRQKLKPYELNPIKTIWGGGYQWRSL
AMBR158 ctg2_142 (core biosynthetic gene = lassopeptide: Aspargine synthase)
SEQ ID No. 10
MKVNVILSKNQIITTDTLIDELVVSEELKTYTADNLLMIVKPHKNSEDIGRFLAKENLNIEMIESNLLELKKNASF
VAITKENHFVIGGDFNNHNPIYYLEKDEELFISFYPELIASFTKQKLNRTYFALRIVNFDTVYPFTELTPWDNVKL
VNSRKILVSLEGQVKQVFPWRSNGENKTIAGVAGELSKNLNSVLRKYLENQPKISADLSGGLDSTSLCYFCKNLGI
ELNTVFLSPEDGADSDVVWSNRASRDVSAEHVILPYSSTVTLSTLDPIAIYNALPFGPSESFRYINLARKIIEIGL
ENDISTHINGHGGDELFGPLSTMAWSLYHSNYPNRIRNLYGFARLNKFNLFKFFKSLTDNTSLSDNLRNLVCQEVE
QVDFDIKYSSKWISTLSVESFVTQTARDLITRCATSYAEKDIESYSDDKSIHKILEEIESHTLLQRDLNAMVPTDK
YFRFLSPYTNYDIVESALKYNIDERFNAKVTKPMLAEIRPSSMSLEYFQRRDKGEYSKPTFTEYSKRKKILQKIFS
EECLIYKLGLVDRVDLLESLNKFSADGELLENVMRLQIIEFWLRGYCESEGEYEI
AMBR158 ctg2_144 (core biosynthetic gene = Lassopeptide biosynthesis B2
protein) - SEQ ID No. 11
MSSPVRMEQRTKLSVKNKITALFCANVSFFLIKLPPKKLSEVIEKLSKNTRKALPKEVESWRTSINSINVRCAGNG
CLQRSVAVMLRGIIARRTPDWVSGFQVSPFIAHAWVEVDGIPIGEEMDLSNFQKILFVKGGQR
AMBR158 ctg2_145 (transport-related gene = ABC transporter yxLF) - SEQ ID
No. 12
MIKIENVSKAFREKIVLSKISFTANEGEVTALIGPNGSGKSTLLKILLGLISTDEGRATFDGKNYGELGKYPFRYV
GTFLDSFQPNPTRTAYNHLRWIALTSGISKQRCLECLKIVGLQDVGNKKIKDYSLGMKQRLGLATAILANPKILIL
DEPINGLDPDGIRWVREFLREFVKDDKIVLITSHYMNELELTVDKIVGLSNGKVVIDGSSKDVLEEYGSFEEAYFR
SVRNEMR
AMBR158 ctg2_148 (transport-related gene = ABC transporter bmrA) - SEQ ID
No. 13
MDSQQHSEVSFREFLRLLKPHKISISIALIIALIASTLSILQPILLSKIIDNINNTILKKTVILFSMIVLSSAILN
SIKQYILESISENLVNSLRIQIVNRSIKYKIEVFDTRKIGDLASILSTDTAQLRGILSQGIVELVSQTFTMLFALI
MMFYLDIQLFSLSLLAVLSLLICGLLLGKRTRPIAKDLQEVVGELSSELERTLRGVRTIRAFLSENVFVERMSSVV
TSATKIGKKVAIFKSIISGFSNIALQIMLIIIIGVGAIRVATGVISIGSLSAFIMYVMLVLTPAAMLGGVLASINE
GLGAYSRIKVALELPIEEDINSLDVLEKKENQILQIKKLSFKYNDNFDKNILNKIDISVNGPGVTALVGPSGSGKT
TIFELIERFYDFDDGTIELYGNNIYDLPIKVLRSNITFVEQNASIFSGTVLENLQIANKNVSRLDCLHALNLVNLF
QDIPSEEILDRVIGESGITLSGGEKQRLSLARALISPNDIILLDETTSNLDSINESKIHQLIKDLSRRKTVLIIAH
RLSTIQNADSIYLISEGEVDDCGTHQELLSRNKLYKELVETQFINKET
BACTERIOCIN LOCUS 2
AMBR158 ctg4_81 (core biosynthetic gene = Lactococcin-G-processing and
transport ATP-binding protein LagD) SEQ ID No. 14
MSKYPYISQVDQRDCGVATLAMILKHYGSSYSLAYLRELAQTSREGTSALGLVEAAKQLGLETQAIRADLDLFKQE
NLSYPFIVHVVKEGGLQHYYTVFGQIKGQLVIGDPDPSKKVIKMPLEEFSKEWTGVALFFVPGEAYTKYKEDVPGL
LSFLPILFRRKGLIAVIVLLSFLVTLVNIIGSYYLQAIIDRLIPQGAYNLLTMISLGLCISYLAQQVFTFFKDYLL
HRLGNYLSIAVILPYIKHVLSLPISFFSSRRTGEITSRFGDANTIIDALASTILSIFLDVTIVMTLAVALIVQNQS
LFLMTLTVVPIYVLIIVAFYKLFEKENYQLMEANSQVNTAVIDDLRGIETLKSLRVEERRYREIEVKFHDYLKKSL
SKAKWQLTQDGLKTGVQLVSNVFILWYGAQLVMEGQLSAGQLITYNMLLNYFTTPLINIINLQSKIQQAKVANNRL
QEVYVVDKEEKGKLRELSFKQLAFKGVSHRFSYQQETLSKIDLTIHKGEKIALMGKSGSGKTTLAKMLSGYYTKSS
GRVTLDKDAISHAELRQLVTYVPQQTYVFTGTILENLLLGFEGEVDEKKLLKVCQQADILEDIQKMPLGFQTQVSE
DGGLSGGQKQRLAIARALLSKQPILIFDEATSGLDSDTESRVMANLAKIKRTMIFIAHRNSVRQHVSRVVTMVSGQ
IESDSPNFNLFQFI

### REFERENCES

1. Schilder A G M, Chonmaitree T, Cripps A W, Rosenfeld R M, Casselbrant M L, Haggard M P, Venekamp R P. 2016. Otitis media. Nat Rev Dis Primer 2:16063.
2. Lous J, Burton M J, Felding J, Ovesen T, Rovers M, Williamson I. 2005. Grommets (ventilation tubes) for hearing loss associated with otitis media with effusion in children, p. CD001801.pub2. In The Cochrane Collaboration (ed.), Cochrane Database of Systematic Reviews. John Wiley & Sons, Ltd, Chichester, UK.
3. Schilder A G M, Manen J G, Zielhuis G A, Grievink E H, Peters S A F, Broek P. 2007. Long-term effects of otitis media with effusion on language, reading and spelling. Clin Otolaryngol Allied Sci 18:234-241.
4. Ngo C C, Massa H M, Thornton R B, Cripps A W. 2016. Predominant Bacteria Detected from the Middle Ear Fluid of Children Experiencing Otitis Media: A Systematic Review. PLoS ONE 11:e0150949.
Kim D, Zeng M Y, Núñz G. 2017. The interplay between host immune cells and gut microbiota in chronic inflammatory diseases. Exp Mol Med 49:e339.
6. Hill C, Guarner F, Reid G, Gibson G R, Merenstein D J, Pot B, Morelli L, Canani R B, Flint H J, Salminen S, Calder P C, Sanders M E. 2014. The International Scientific Association for Probiotics and Prebiotics consensus statement on the scope and appropriate use of the term probiotic. Nat Rev Gastroenterol Hepatol 11:506-514.
7. van den Broek M F L, De Boeck I, Kiekens F, Boudewyns A, Vanderveken O M, Lebeer S. 2019. Translating Recent Microbiome Insights in Otitis Media into Probiotic Strategies. Clin Microbiol Rev 32:e00010-18.
8. Wescombe P A, Heng N C K, Burton J P, Chilcott C N, Tagg J R. 2009. Streptococcal bacteriocins and the case for Streptococcus salivarius as model oral probiotics. Future Microbiol 4:819-835.
9. Kozich J J, Westcott S L, Baxter N T, Highlander S K, Schloss P D. 2013. Development of a Dual-Index Sequencing Strategy and Curation Pipeline for Analyzing Amplicon Sequence Data on the MiSeq Illumina Sequencing Platform. Appl Environ Microbiol 79:5112-5120.
Walker R E, Walker C G, Camargo C A, Bartley J, Flint D, Thompson J M D, Mitchell E A. 2019. Nasal microbial composition and chronic otitis media with effusion: A case-control study. PLoS ONE 14:e0212473.
11. Sanders M E, Benson A, Lebeer S, Merenstein D J, Klaenhammer T R. 2018. Shared mechanisms among probiotic taxa: implications for general probiotic claims. Curr Opin Biotechnol 49:207-216.
12. Dunne E M, Toh Z Q, John M, Manning J, Satzke C, Licciardi P. 2014. Investigating the Effects of Probiotics on Pneumococcal Colonization Using an In Vitro Adherence Assay. J Vis Exp 51069.

1. An isolated bacterial strain of the Streptococcus salivarius (S. salivarius) species, said strain deposited at BCCM under accession number LMG P-31813.

2. The isolated bacterial strain according to claim 1, wherein the bacteria are in suspension, freeze-dried, spray-dried in activated or inactivated form, provided that they are not killed.

3. One or more secondary metabolites produced by the isolated bacterial strain according to claim 1 or 2.

4. A composition comprising an isolated bacterial strain of the Streptococcus salivarius (S. salivarius) species according to claim 1 or 2, and/or one or more secondary metabolites according to claim 3.

5. The composition according to claim 4 comprising one or more pharmaceutically acceptable excipients, aromatizing agents or carriers.

6. The isolated bacterial strain according to claim 1 or 2, the secondary metabolites according to claim 3 or the composition according to claim 4 or 5 for use as a medicament in human or veterinary medicine.

7. The isolated bacterial strain, the secondary metabolites or the composition according to claim 6, for use in the treatment and/or prevention of infections and/or inflammatory diseases.

8. The isolated bacterial strain, the secondary metabolites or the composition for use according to claim 7 wherein the infections and inflammatory diseases are selected from: upper respiratory tract infections; ear, nose, and throat (ENT) infections; oral cavity infections; throat infections, caries; sinusitis; nasal polyposis; acute otitis media; recurrent otitis media; otitis media with effusion; chronic suppurative otitis media; mastoiditis; halitosis; respiratory infections associated with cystic fibrosis.

9. The isolated bacterial strain, the secondary metabolites or the composition for use according to claim 8 in the treatment of otitis media with effusion.

10. The isolated bacterial strain, the secondary metabolites or the composition according to claim 6, for use in immunomodulation.

11. The isolated bacterial strain, the secondary metabolite or the composition for use according to claim 10 wherein the isolated bacterial strain, the secondary metabolites or the composition is used as an adjuvant to promote an immune response during vaccination.

12. Use of the isolated bacterial strain according to claim 1 or 2, the secondary metabolites according to claim 3 or the composition according to claim 4 or 5 in personal hygiene industry, cleaning industry, air purification, or the production of personal care/consumer product; preferably in personal oral hygiene industry.

13. Use of the isolated bacterial strain according to claim 1 or 2, the secondary metabolites according to claim 3, or the composition according to claim 4 or 5 as a probiotic.

14. The use of the isolated bacterial strain, the secondary metabolites or the composition according to claim 12 in food industry; in particular in the production of dairy and non-dairy fermentation production, dietary supplements, dietary food additives, nutraceuticals.

15. The isolated bacterial strain, the secondary metabolite or the composition according to any one of claims 1 to 11 or the use according to any one of claims 12 to 14, wherein the isolated bacterial strain, the secondary metabolite or the composition is in any form suitable to be administered topically, orally or through the respiratory tract.
